(19) United States
(12) Patent Application Publication
(10) Pub. No.: US 2009/0221622 A1
(43) Pub. Date: Sep. 3, 2009

(54) TOPOTECAN READY TO USE SOLUTIONS

(75) Inventors: Bulusu Bhanu TEJA, Hyderabad (IN); Philip Christopher BUXTON, Great Dunmow (GB); Nagesh R. PALEPU, Southampton, PA (US)

Correspondence Address: LUCAS & MERCANTI, LLP, 475 PARK AVENUE SOUTH, 15TH FLOOR, NEW YORK, NY, 10016, US

(73) Assignee: EAGLE PHARMACEUTICALS, INC., Woodcliff Lake, NJ (US)

(21) Appl. No.: 12/394,431

(22) Filed: Feb. 27, 2009

Related U.S. Application Data
(60) Provisional application No. 61/032,652, filed on Feb. 29, 2008.

(51) Int. Cl. A61K 31/437

(52) U.S. Cl. 514/283

(57) ABSTRACT

Aqueous-based, ready to use topotecan-containing formulations for parenteral use having extended stability are disclosed. The formulations are surprisingly free of precipitated degradation products such as 10-hydroxycamptothecin (10-HCPT) after periods of up to 1 year or greater.

### CROSS-REFERENCE TO RELATED APPLICATIONS

This application claims priority under 35 U.S.C. §119(e) to U.S. Provisional Patent Application No. 61/032.652. filed Feb. 29, 2008, entitled “TOPOTECAN READY TO USE SOLUTIONS,” the disclosure of which is incorporated by reference herein in its entirety.

### BACKGROUND OF THE INVENTION

Camptothecin is a water-insoluble, cytotoxic alkaloid produced by Camptotheca accuminata trees indigenous to China and Nothapodytes foetida trees indigenous to India. Camptothecin and a few close congeners thereof are the only class of compounds known to inhibit topoisomerase I.

Inhibition of topoisomerase II is the major target of important commercial oncolytic agents (e.g., etoposide) as well as other oncolytic agents still undergoing development. Camptothecin (and its known congeners) have no effect on topoisomerase II and none of the known topoisomerase II inhibitors has any significant effect on topoisomerase I.

Camptothecin and its known topoisomerase I inhibiting congeners have not proven to be attractive for clinical drug development as cytotoxic agents due to unacceptable dose limiting toxicity, poor aqueous solubility, and/or unacceptable shelf life stability. Therefore, there is a need for topoisomerase I inhibiting agents which avoid the undesirable features of camptothecin and its known related topoisomerase I inhibiting congeners.

Topotecan is a water soluble conjugate of Camptothecin marketed under the trade name Hycamtin as a lyophilized powder for injection. The dosage strength is 4 mg free base per vial and is intended as a single use vial. The recommended dose is 1.5 mg/m² of intravenous infusion over 30 minutes daily for 5 consecutive days starting on day 1 of a 21-day course. In the absence of tumor progression, a minimum of 4 doses is recommended. The median time to response in 3 ovarian cancer trials was 9 to 12 weeks, and the median time to response in 4 small cell lung cancer cell trials was 4 to 7 weeks. The dose limiting toxicity is neutropenia (bone marrow suppression) and thrombocytopenia.

Hycamtin should be reconstituted with 4 ml of water for injection and under a vertical laminar flow hood while wearing gloves and protective clothes. Then, the appropriate volume of reconstituted solution is diluted into either normal saline or 5% dextrose solution prior to administration. Since the lyophilized dosage form contains no antibacterial preservative, the reconstituted product should be used immediately.

The major disadvantage with the lyophilized product is that it is a single strength vial of inappropriate dose. For example, the normal body surface area of cancer patients varies Camptothecin and a few close congeners thereof are the only class of compounds known from 1 to 2 square meters which corresponds to a dose of 1.5 to 3 mg of topotecan. For each reconstituted vial, there will be wastage of 1 to 2.5 ml if the reconstituted solution is used for a single patient per day. Therefore, the overall wastage per cycle per patient is anywhere from 5 to 12.5 ml. In addition, disposing of this solution can be quite expensive.

Ideally, a ready-to-use solution that is either self preserved or contains an antibacterial agent would allow dose flexibility and reduce wastage of solution. A ready to use solution also would protect the dispensing pharmacist from exposure to drug during the reconstitution. Generally the lyophilized product produces aerosol particles during the reconstitution which are potentially harmful to the operator.

Such ready to use solutions are not currently available because of the poor solution stability of Topotecan. The lactone structure renders the molecule susceptible to hydrolysis in the mid to alkaline region of the pH range. Kearney A. S. et al. in “Preformulation Studies to Aid in the Development of a Ready-To-Use Injectable Solution”, Intl. J. Pharmaceutics 127 (1996) 229-237, report on the stability of topotecan under acidic conditions in the range of pH 2.5-4. The process of degradation is a deamination via formation of a reactive quinone methide from the zwitterionic species of topotecan. The reactive quinone methide further hydrolyses to form 9-methylhydroxy-10-hydroxy camptothecin. In a subsequent acid catalyzed step, the formation of 10-hydroxycamptothecin (10-HCPT) occurs via loss of formaldehyde from the intermediate. This degradation product is especially troublesome in the design of ready-to-use (hereinafter, “RTU”) formulations. The 10-HCPT is extremely poorly soluble in aqueous media (about 2 ng/ml). Thus, in currently known aqueous formulations, it quickly appears as a precipitate or as crystals in the vials containing the topotecan solution. Vials containing such degradation crystals would have to be discarded at considerable effort and expense since they are unsuitable for further administration to patients. The crystals are a danger to patients and can cause significant injury, i.e. burn, if extrasavated. The insoluble crystals can also cause problems if they are trapped in the artery during administration. Thus, there is a continued need for topotecan formulations which are RTU and have long term stability. The present invention addresses this need.

### SUMMARY OF THE INVENTION

The invention is generally directed to aqueous-based topotecan-containing compositions that are stable for extended periods of time when stored at room temperature. In most aspects, this will include temperatures of less than or equal to about 25° C. The inventive compositions are substantially free of precipitated 10-HCPT, even after periods of storage of one year or longer at these temperatures. In some preferred aspects of the invention, the amount of topotecan or one of its pharmaceutically acceptable salts included in compositions is from about 1 to about 5 mg/mL, expressed as the free base. The inventive solutions also preferably contain a pharmaceutically acceptable strong acid such as hydrochloric acid (HCl) or methanesulfonic acid (MSA) or trifluoro acetic acid (TFA) of normality and/or strength sufficient to yield a topotecan-containing solution of acidity less than or equal to about pH 1.5, and in more preferred embodiments, less than or equal to about 1.2.

In another embodiment, the inventive solutions can include alternative pharmaceutically acceptable acids having a pKa higher than that associated with the strong acids defined herein. The acids selected provide a sufficient increase in the solubility of the 10-HCPT so that the amount of 10-HCPT formed during long term storage does not exceed the solubility of the 10-HCPT for the solution. Suitable alternative acids include those such as hydroxyl acids other than dihydroxy dicarboxylic acids or dihydroxy succinic acids, such as tartaric acid. Other suitable alternative acids include hydroxyl tricarboxylic acids, such as citric acid, hydroxy carboxylic acids, such as 5 to 50% lactic acid or similar acids capable of maintaining the pH of the inventive compositions at or below the levels described herein, i.e. 1.5 or alternatively 1.2. These alternative acids can be used in addition to or in place of some or all of the “strong” acids mentioned above.

In accordance with some particularly preferred embodiments, stable compositions according to the invention include those in which: a) the topotecan maintains a concentration greater than about 95% of the original amount (i.e. label) claim for a period of at least about 12 months at a temperature of less than or equal 25° C.; and/or b) the formulation is substantially free of precipitated 10-HCPT for a period of at least 12 months at temperatures of less than or equal to about 25° C., hereinafter “room temperature” or for two months storage at 40° C. It is appreciated by those of ordinary skill that room temperature will vary, depending upon location, time of year, etc., and that the compositions of the invention also demonstrate improved long term stability even when non-climate controlled environments exceed 25°, i.e. up to 30° C. or greater.

The above-mentioned Kearney et al. publication reports stability data in a pH range some three to four units below the pKa values of the phenolic and dimethylamino centers of topotecan which would be expected to be almost fully protonated, greater than 99.9%. Consequently there would not be expected to be much improvement in chemical stability on reducing the pH significantly below those mentioned therein, let alone by a further one or two units. Surprisingly, it has now been discovered that the stability of topotecan, as measured at least in one aspect by the absence of the precipitated, degradant 10-HCPT, is much improved by reducing the pH of the topotecan-containing solutions to pH levels of 1.5 or less, and preferably 1.2 or less. Moreover, it has also been surprisingly found that the solubility of 10-HCPT is increased at lower pH's. Thus, the 10-HCPT which results from the degradation of the topotecan during long term storage does not precipitate in the pharmaceutically suitable fluid until it reaches a concentration of about 4 μg/mL when the formulations are prepared in hydrochloric acid in accordance with the description provided herein, especially with regard to pH. As a result, when appropriate storage conditions are employed, i.e. room temperature or under refrigerated conditions, a viable product shelf-life for an aqueous RTU product is attained. For instance, a 1 mg/ml solution of topotecan at pH 3.2 degraded over five weeks at 40° C. to give 5.64% of 10-HCPT whereas similar solutions at about pH 2 and pH 1 over twelve weeks at the same temperature yielded only 0.6% and 0.07% of 10-HCPT respectively. Thus, these accelerated stability data confirm that the inventive topotecan solutions have shelf lives of a year or more when kept at room temperature.

Optional aspects of the invention include the addition benzyl alcohol at a concentration of up to 3%. It has been surprisingly found that the addition of the benzyl alcohol in formulations maintained at low pH's substantially enhances the solubility of the 10-HCPT. Indeed, the solubility of the 10-HCPT was found to go from about 2 ng/mL to about 10 μg/mL in 0.01N HCl containing 3% benzyl alcohol, i.e. about a 5000-fold increase, within the pH levels recited herein.

Still further aspects of the invention include methods of treatment using the inventive topotecan-containing compositions, kits containing the same and methods of preparing the compositions described herein.

### DETAILED DESCRIPTION OF THE INVENTION

In accordance with one aspect of the invention there are provided topotecan-containing compositions which have improved long term stability at room temperature. In one embodiment, the invention includes topotecan-containing compositions include:
a) topotecan or a pharmaceutically acceptable salt thereof; and
b) a pharmacologically suitable fluid comprising an aqueous hydrochloric acid diluent, wherein:
   i) the pH of the composition is less than or equal to about 1.5; and
   ii) the composition is stable during long term storage;
   wherein the amount of 10-hydroxycamptothecin (10-HCPT) in the composition resulting from the degradation of said topotecan during said long term storage is less than about 6 μg/mL.

Preferably, the 10-HCPT in the composition is less than about 2-4 μg/mL. More preferably, the amount of 10-HCPT is less than about 1-2 μg/mL.

In a second embodiment, the topotecan-containing compositions include:
a) topotecan or a pharmaceutically acceptable salt thereof; and
b) a pharmacologically suitable fluid comprising an aqueous hydrochloric acid diluent, wherein:
   i) the pH of the composition is less than or equal to about 1.5; and
   ii) the composition is stable during long term storage;
   wherein the 10-hydroxycamptothecin (10-HCPT) resulting from the degradation of said topotecan during said long term storage does not precipitate in said pharmaceutically suitable fluid until the 10-hydroxycamptothecin (10-HCPT) reaches a concentration of greater than about 6 μg/mL.

In a third embodiment, the topotecan-containing compositions include:
a) topotecan or a pharmaceutically acceptable salt thereof; and
b) a pharmacologically suitable fluid comprising lactic acid, wherein:
   i) the pH of the composition is greater than or equal to about 1.5; and
   ii) the composition is stable during long term storage;
   wherein the 10-hydroxycamptothecin (10-HCPT) resulting from the degradation of said topotecan during said long term storage does not precipitate in said pharmaceutically suitable fluid until the 10-hydroxycamptothecin (10-HCPT) reaches a concentration of greater than about 10 μg/mL.

In yet another preferred embodiment there is provided a topotecan-containing composition which includes:
a) topotecan or a pharmaceutically acceptable salt thereof; and
b) a pharmacologically suitable fluid comprising an aqueous hydrochloric acid diluent, wherein:
   i) the pH of the composition is less than or equal to about 1.2;
   ii) the composition is stable during long term storage; and
   iii) the topotecan concentration is about 2 mg/mL to about 4 mg/mL;
   wherein the amount of 10-hydroxycamptothecin (10-HCPT) in said composition resulting from the degradation of said topotecan during said long term storage is less than about 6 μg/mL.

In yet another embodiment there is provided a topotecan-containing composition which includes:
a) topotecan or a pharmaceutically acceptable salt thereof; and
b) a pharmacologically suitable fluid comprising lactic acid, wherein:
   i) the pH of the composition is less than or equal to about 1.2;
   ii) the composition is stable during long term storage; and
   iii) the topotecan concentration is about 2 mg/mL to about 4 mg/mL;
   wherein the amount of 10-hydroxycamptothecin (10-HCPT) in said composition resulting from the degradation of said topotecan during said long term storage is less than the solubility of the 10-HCPT in that composition or, less than about 10 μg/mL.

In each of the embodiments described herein, the amount of 10-HCPT in the compositions of the present invention does not exceed the solubility of the 10-HCPT during the period of storage. Thus, the compositions have extended stability, i.e. a shelf life of several months or longer, at room temperature.

The topotecan compositions of the present invention are also preferably formulated at a concentration to dilute in the infusion bags of 50 to 250 ml for infusion to a mammal in need thereof. For purposes of the present invention, such compositions are deemed to be “ready to use” or RTU because they are fit for direct dilution into infusion bags.

As will be appreciated by those of ordinary skill, since the formulations of the present invention are designed for parenteral use, the inventive compositions are sterile or sufficiently sterile to meet all United States Pharmacopeia and FDA requirements for dosage forms of this type.

In some preferred aspects of the invention, the pH of the composition is less than or equal to about 1.2. In other preferred embodiments, the pH is between about 1 and about 1.2. In some other embodiments, the pH is between 1.4 to 1.7. In order to maintain the pH at the desired levels, the composition, actually a sterile solution, preferably contains a pharmaceutically acceptable acid in sufficient quantity and strength to yield a solution acidity of at least less than or equal to about pH 1.7, and preferably less than or equal to 1.2. More preferably, the amount and strength of the acid or a combination of acids thereof is sufficient to maintain the pH within the range of about 1 and 1.7. Suitable acids include those generally accepted by those of ordinary skill as being pharmaceutically acceptable as such term is understood in the art. A non-limiting list of such acids include, but not exclusively, hydrochloric acid (HCl), methanesulfonic acid (MSA) or other strong acids such as sulfuric acid, trifluoro acetic acid or any strong acid with pKa<1.0. In some preferred aspects of the invention, the acid is HCl or MSA.

In alternative aspects of the invention, the topotecan-containing compositions of the invention can include an acid other than those “strong” acids described above. These alternative acids can replace a portion or all of the strong acid provided, however, that they are included in sufficient strength and concentration to maintain the pH of the topotecan-containing composition below the thresholds described herein, i.e. 1.5 or 1.2. Generally, suitable alternative acids will be well-known to those of ordinary skill because of their known utility as pharmaceutically acceptable acids. A non-limiting list of suitable acids in this aspect of the invention therefore include acids such as phosphoric, lactic, citric, acetic and the like. Mixtures of the preferred strong acids and the alternative acids are also contemplated so long as the desired pH levels are maintained. Regardless of the aspect of the invention, a solution acidity of at least less than or equal to about pH 1.7, and preferably less than or equal to 1.2 is used in order to obtain compositions having the extended stability and shelf life described herein. More preferably, the type and amount of acid is sufficient to maintain the pH within the range of about 1 and 1.2.

The amount of acid included in the compositions of the present invention can readily be adjusted based on the desired normality or strength in order to keep the pH of the product within the desired range.

For purposes of the present invention, “long term storage” shall be understood to include at least time periods which are in excess of those observed when currently available lyophilized topotecan formulations are reconstituted. In some preferred aspects of the invention, the time for which long term storage are contemplated include periods of at least about 3 to 4 months. In other preferred aspects, the periods include at least about 52 weeks, more preferably, at least about 78 or at least about 104 weeks.

For purposes of the present invention, “stable” shall be understood to mean that the compositions of the present invention maintain at least about 95% of the initial amount of topotecan after the storage period. Stated another way, the potency loss of the active material, topotecan, is less than about 5% after the storage periods described herein. Stable shall also be understood to mean that the total amount of 10-HCPT present in the compositions of the invention in the dissolved form is less than about 6 μg/ml in hydrochloric acid solutions and less than 10 μg/ml in aqueous lactic acid solution, when measured at any time during the period of long term storage. Preferably, the amount of soluble form of 10-HCPT in the compositions of the present invention is less than about 2-4 μg/ml and in another embodiment, it is less than about 1-2 μg/ml for the topotecan compositions hydrochloric acid solution.

For purposes of the present invention, a pharmacologically suitable fluid comprising an aqueous diluent shall be understood to include all known fluids capable of being included in sterile parenteral formulations. Such aqueous-based suitable fluids can include, for example, saline or dextrose if desired as well any of the known ancillary preservatives or excipients commonly found as part of parenteral formulations. In accordance with current FDA requirements, vials containing the inventive formulations contain well below the acceptable limits for particulate matter. Thus, the vials contain:

Particles≧10 μm: Not more than 6000 per container (average)

Particles≧25 μm: Not more than 600 per container (average)

The temperatures in which the compositions are preferably kept are said to be either room temperature or less (i.e., about 25° C. or less). While not required, it is contemplated that storage can be further increased if carried out (optionally) under refrigerated conditions.

For purposes of the present invention, “refrigerated conditions” shall be understood as being temperature below room temperature and preferably temperatures of less than about 10° C., preferably from about >0° C. to about 10° C., more preferably from about 2 to about 10° C., yet more preferably about 3 to about 8° C., and still more preferably about 5° C. The term “refrigerated” conditions shall further be understood as including maintaining the composition at a substantially constant temperature and storage conditions within this range.

Thus, in those aspects of the invention where the RTU topotecan compositions of the present invention are stored at room temperature, they remain in the temperature range described herein for substantially the entire period between shortly (generally no more than a few hours) after manufacture and shortly (generally no more than a few hours) before dilution and administration to the patient in need thereof.

The compositions of the present invention preferably include topotecan. It is understood by those of ordinary skill, however that the invention includes pharmaceutically acceptable salts, prodrugs or solvates thereof. The invention is therefore described in terms of topotecan and the concentration of the drug in the compositions is expressed as the free base. It will be understood that when these alternative forms are included, the amount of active is nonetheless calculated on the basis of the free base. Preferred topotecan concentrations are from about 1 mg/mL to about 5 mg/mL. Alternative concentrations include from about 2 mg/mL to about 4 mg/mL, or about 3 mg/mL. The inventive formulations can be in single use or multiple-use vials.

In an alternative aspect of the invention, the compositions optionally include benzyl alcohol in amounts of up to about 3% by weight. A preferred embodiment of this aspect includes up to about 1% by weight benzyl alcohol. In these aspects of the invention, the amount of precipitated 10-HCPT is therefore negligible. The amount of 10-HCPT in solution, however, can be up to about 10 μg/mL without deleterious effect on the treatment of the patient receiving the topotecan therapy. Therefore, in this alternative aspect of the invention, there is provided a topotecan-containing composition, comprising:
a) topotecan, or a pharmaceutically acceptable salt thereof;
b) benzyl alcohol; and
c) a pharmacologically suitable fluid comprising an aqueous diluent, wherein:
   i) the pH of the composition is less than or equal to about 1.5; and
   ii) the composition is stable during long term storage and substantially free of precipitated 10-hydroxycamptothecin (10-HCPT) during the long term storage.

For purposes of the present invention, “substantially free” shall be understood to mean an amount which is less than that detectable which is detectable by the eye upon visible inspection. In addition, compositions are said to be “substantially free” of precipitated 10-HCPT if they contain less than the aforementioned FDA limits for particulate matter in the vials containing the aqueous topotecan compositions.

An alternative embodiment of the invention includes multi-dose vials containing a 2-4 mg/ml topotecan solution. Such vials can contain up to 30 ml or smaller volumes, if desired. This is to be contrasted to some embodiments in which a single dose vial containing the inventive compositions contains 1 or 2 ml of 2-4 mg/ml solution or a pharmaceutically acceptable salt thereof. In either case, the compositions in the vials have improved long term storage. Thus, a single vial can be used to administer topotecan to multiple patients, thereby lessening wastage. More importantly, a single multi-dose vial can be used to administer an entire set of 5 daily doses to a single patient or multiple patients or even an entire 28 day cycle to a single or multiple patients and thereby substantially lessen wastage even where few patients or only a single patient is in need of topotecan administration. As used herein, a multi-dose vial includes vials in which the medication can be used for 28 days after first insertion. It allows hospital staff to withdraw solution up to 10-15 times from the vial. For example a single dose vial will be only 1-2 ml of 3 mg/ml solution and after administering the patient, the remaining solution is discarded. The multi-dose vial can be up to 15 ml of 3 mg/ml solution per vial. This vial can be can be used to administer the drug to 10 to 15 patients over 28 days. Other such multi-use vials can include amount of from 2 to 15, or greater, if desired.

A further aspect of the invention includes a kit containing the topotecan-containing compositions described herein. As will be appreciated by those of ordinary skill, the kit will contain at least one pharmaceutically acceptable vial or container containing one or more doses of the topotecan-containing compositions as well as other pharmaceutically necessary materials for storing and/or administering the drug, including instructions for storage and use, infusion bag or container with normal saline or D₅W, additional diluents, if desired, etc.

The compositions of the present invention can be packaged in any suitable sterile vial or container fit for the sterile storage of a pharmaceutical such as topotecan. Suitable containers can be glass vials, polypropylene or polyethylene vials or other special purpose containers such as CZ vials manufactured by Diakyo or glass vials with quartz-like inner surfaces manufactured by Schott.

In yet another aspect of the invention there are provided methods of treating a topotecan sensitive disease in mammals. The methods include administering an effective amount of a topotecan-containing composition as described herein to a mammal in need thereof. Since the active ingredient portion of the inventive compositions is an FDA-approved drug, those of ordinary skill will recognize that the doses of topotecan employed in this aspect of the invention will be the similar to those employed in any treatment regimens designed for topotecan as marketed under the trade name Hycamtin. The patient package insert containing dosing information is incorporated herein by reference. The methods of treatment also include administering the inventive formulations for any purpose or physical condition for which topotecan is has been indicated as being useful.

A still further aspect of the invention includes methods of preparing the topotecan compositions described herein. The methods include dissolving topotecan or pharmaceutically acceptable salt thereof in a sufficient amount of an aqueous solution containing a sufficient amount of an acid under conditions sufficient to dissolve the topotecan while maintaining the pH of the resulting solution at about 1.5, and preferably 1.2 or less.

In a further aspect of the invention, there is provided a method of preventing the formation of precipitated 10-hydroxycamptothecin in topotecan-containing aqueous formulations during long term storage at room temperature. The method includes forming an aqueous-based topotecan formulation at a concentration of up to about 5 mg/ml and thereafter adjusting the pH the aqueous formulation less than or equal to about 1.5 prior to initiating the long term storage.

### EXAMPLES

The following examples serve to provide further appreciation of the invention but are not meant in any way to restrict the effective scope of the invention.

### Preparation

### Example 1

Topotecan was dissolved in hydrochloric acid solution (0.1 M) to a concentration of 1 mg/mL. The acidity was approximately pH 1. The solution was filled into flint glass vials sealed with caps and stored at 40° C. and assayed for 4, 8 and 12 weeks by a stability indicating HPLC method which measures both topotecan and 10-HCPT. After 12 weeks storage, the reaction solution was clear and there was no evidence or presence of any precipitate or crystals. The solution was analyzed and showed a topotecan concentration of 100% of the initial value and a 10-HCPT content of 0.07% equivalent to a concentration of 0.7 μg/mL. Quantitatively, 0.01 area % is equivalent to 0.1 μg/mL of 10-HPCT. The amount of 10-HCPT is well below the solubility of 10-HCPT in 0.1 N HCl which is 6 μg/mL. This accelerated data indicates that such a product will have a shelf-life at room temperature, e.g. about 25° C., in excess of 18 months. The stability data is presented in the Table 1 below:

TABLE 1
Stability of topotecan (1 mg/ml) in 0.1N HCl (Acidity ~pH 1)

| | | | | |
|---|---|---|---|---|
| Storage | Time | Conc. | % of | Concentration of |
| Temp. | period | mg/mL | Initial | 10-HCPT (μg/ml) |

| | | | |
|---|---|---|---|
| Initial | 1.00 | 100 | None |

| | | | | |
|---|---|---|---|---|
| 40° C. | 2 weeks | 1.00 | 100 | None |
| | 4 weeks | 1.00 | 100 | 0.4 |
| | 8 weeks | 1.00 | 100 | 0.6 |
| | 12 weeks | 1.00 | 100 | 0.7 |

### Example 2

The topotecan preparation of 1 Ai was made with a concentration of 3 mg/mL topotecan instead of 1 mg/mL. The solution was filled into either flint glass vials or polyethylene vials. One portion of the solution was transferred into 5 ml glass vials and another portion was placed into 5 ml PE vials. The vials were then subjected to accelerated stability testing and the stability data is presented in the Table 2 below:

TABLE 2
Stability of topotecan (3 mg/ml) in 0.1N HCl (pH 1.19)

| | | | | | Concentration |
|---|---|---|---|---|---|
| Lot ID | Storage | Time | Content | % of | of 10-HCPT |
| TPT #1 | Temp. | Period | mg/mL | Initial | (μg/ml) |
| (Flint | Initial | | 3.07 | 100 | 1.0 |
| Vial) | 40° C. | 1 month | 3.06 | 100 | 0.92 |
| | | 2 months | 3.06 | 100 | 1.54 |
| | | 3 months | 3.04 | 99 | 2.15 |
| | | 6 months | 2.98 | 97 | 3.99 |
| | 25° C. | 3 months | 3.07 | 100 | 0.061 |
| | | 6 months | 3.04 | 99 | 0.06 |
| | | 12 months | 3.05 | 99 | 1.23 |
| (PE Vial) | Initial | | 3.06 | 100 | 1.0 |
| | 40° C. | 1 month | 3.06 | 100 | 0.61 |
| | | 2 months | 3.05 | 100 | 1.53 |
| | | 3 months | 3.05 | 100 | 2.14 |
| | | 6 months | 2.98 | 97 | 3.67 |
| | 25° C. | 3 months | 3.05 | 100 | 0.61 |
| | | 6 months | 3.04 | 99 | 0.61 |

The solutions from each vial were analyzed and each showed a topotecan concentration of at least 97% of the initial value and a 10-HCPT content of no more than 3.99 μg/mL after 6 months. The amount of 10-HCPT formed is well below the 6 μg/mL solubility of 10-HCPT in 0.1 N HCl. These accelerated data indicate that topotecan products will have a shelf-life at room temperature, e.g. about 25° C., in excess of 24 months and at least 3 years regardless of the vial used for storage.

### Example 3

The topotecan preparation of Example 1 was made in methanesulfonic acid instead of HCl acid solution (0.1 N). The stability data is presented in the Table 3 below:

TABLE 3
Stability of topotecan (1 mg/ml) in 0.1N Methanesulfonic acid

| | | | | Concentration |
|---|---|---|---|---|
| Storage | | Conc. | | of 10-HCPT |
| Temp. | Time period | mg/mL | % of Initial | (μg/ml) |
| Initial | | 1.01 | 100 | None |
| 40° C. | 2 weeks | 1.02 | 101 | None |
| | 4 weeks | 1.02 | 101 | 0.4 |
| | 8 weeks | 1.01 | 100 | 0.7 |
| | 12 weeks | 1.00 | 98 | 0.8 |

As was the case with Example 1, the accelerated data indicates that such a product will have a shelf-life at room temperature or about 25° C. in excess of 18 months. The solution was analyzed and showed a 10-HCPT content of 0.08% at 12 weeks, equivalent to a concentration of 0.8 μg/mL, which is well below the solubility of 10-HCPT in 0.1 N MSA which is about 6 μg/mL.

### Example 4

The topotecan composition of 3 mg/ml was prepared in 0.05N hydrochloric acid solution, resulting in a pH of 1.38. The resultant product was subjected to stability testing as in the case of Examples 1-3.

TABLE 4
Stability of topotecan (3 mg/ml) in 0.05N HCl (pH 1.38)

| | | | | |
|---|---|---|---|---|
| | | | | Concentration |
| Storage | Time | Content | % of | of 10-HCPT |
| Temp. | Period | mg/mL | Initial | (μg/ml) |

| | | | |
|---|---|---|---|
| Initial | 3.04 | 100 | 0.9 |

| | | | | |
|---|---|---|---|---|
| 40° C. | 1 month | 3.04 | 100 | 1.52 |
| | 2 months | 3.08 | 101 | 2.74 |
| | | pH 1.38 | | |
| | 3 months | 3.05 | 100 | 4.56 |
| | | pH 1.42 | | |
| 25° C. | 3 months | 3.03 | 100 | 0.91 |
| | 6 months | 3.00 | 99 | 1.22 |
| | 12 months | 3.07 | 101 | 2.13 |

The data presented in Table 4 indicates that such a product will have a shelf-life at room temperature or about 25° C. for at least 2 years.

### Comparative Example 5

The following topotecan compositions of 3 mg/ml were prepared in different strengths of lactic acid solution, resulting in a pH range of 1.5 to 1.7.

TABLE 5
Stability data of Topotecan solution (3 mg/ml) in 10%
lactic acid, pH 1.72

| | | | | Concentration | |
|---|---|---|---|---|---|
| Storage | Time | Content | % of | of 10-HCPT | |
| Temp. | Period | mg/mL | Initial | (μg/ml) | pH |
| Initial | | 3.07 | 100 | | 1.70 |
| 40° C. | 1 month | 3.08 | 100 | 4.30 | ND |
| | 2 months | 3.11 | 101 | 9.21 | 1.74* |
| | 3 months | 3.03 | 99 | 12.0 | 1.80* |

*particles observed due to the precipitation of 10-HCPT
ND: not determined

The saturation solubility of 10 HCPT in 10% lactic acid is 6.4 μg/ml, particulate matter was observed in two and three month's stability samples. Crystals were observed because the amount of 10 HCPT formed in two and three months stability testing exceeded the saturation solubility of 10 HCPT. Therefore, it can be concluded that the topotecan formulation in 10% lactic acid is not suitable for use in long term storage compositions containing topotecan.

### Example 6

The following topotecan compositions of 3 mg/ml were prepared in glass vials in the same manner as Comparative Example 5, except in a 15% lactic acid solution. The stability data are summarized in the Table 6 below.

TABLE 6
Stability data of Topotecan solution (3 mg/ml)
in 15% lactic acid, pH 1.64

| | | | | Concentration | |
|---|---|---|---|---|---|
| Storage | Time | Content | % of | of 10-HCPT | |
| Temp. | Period | mg/mL | Initial | (μg/ml) | pH |
| Initial | | 3.13 | 100 | | 1.62 |
| 40° C. | 1 month | 3.09 | 99 | 3.44 | ND |
| (Flint | 2 months | 3.09 | 99 | 7.51 | 1.61 |
| vials) | 3 months | 3.10 | 99 | 9.70 | 1.66 |

The amount of 10-HCPT formed from topotecan solution is lower in 15% lactic acid compared to that of 10% lactic acid at one, two and three months time period. Precipitation of 10-HCPT at the end of three months storage period was not observed. Therefore the RTU solution of topotecan in 15% lactic acid solution is suitable for long term storage. The pH change during the 3-month storage is insignificant. We have also tested the same formulation in polypropylene vials. The stability data are summarized in the Table 7 below.

TABLE 7
Stability data of Topotecan solution (3 mg/ml) in 15% lactic acid,
pH 1.64

| | | | | |
|---|---|---|---|---|
| | | | | Concentration |
| Storage | Time | Content | % of | of 10-HCPT |
| Temp. | Period | mg/mL | Initial | (μg/ml) |

| | | | |
|---|---|---|---|
| Initial | 3.11 | 100 | = |

| | | | | |
|---|---|---|---|---|
| 40° C. | 1 month | 3.13 | 101 | 3.42 |
| (PP vial) | 2 months | 3.09 | 99 | 7.46 |
| | 3 months | 3.07 | 99 | 9.64 |

### Example 7

The following topotecan compositions of 3 mg/ml were prepared in glass vials in the same manner as Comparative Example 6, except in a 20% lactic acid solution. The stability data is presented in the Table 8 below:

TABLE 8
Stability data of Topotecan solution (3 mg/ml)
in 20% lactic acid, pH 1.54

| | | | | Concentration | |
|---|---|---|---|---|---|
| Storage | Time | Content | % of | of 10-HCPT | |
| Temp. | Period | mg/mL | Initial | (μg/ml) | pH |
| Initial | | 3.10 | 100 | — | 1.57 |
| 40° C. | 1 month | 3.09 | 100 | 2.79 | ND |
| | 2 months | 3.08 | 99 | 6.2 | 1.58 |
| | 3 months | 3.06 | 99 | 8.06 | 1.60 |

The solubility of 10 HCPT in 20% lactic acid solution is 15 μg/ml. The amount of 10-HCPT formed at the end of three months storage at 40° C. was 50% below the saturation solubility of 10-HCPT. Therefore the RTU solution of 3 mg/ml topotecan in 20% lactic acid solution is suitable for long term storage. This solution formulated with Lactated Ringers (1 to 50 dilution) will yield a solution of pH about 4.0.

The data presented in Tables 6 and 7 show that RTU solutions of topotecan are suitable for long term storage when the lactic acid strength is greater than 10%.

### Example 8

The Topotecan compositions of the above 1A-1D are prepared as before except that they are formulated by adding 1 or 3% benzyl alcohol to the composition.

### Additional Examples

Further Examples have been prepared to demonstrate the stability of topotecan formulations in 2M lactic, citric and tartaric acid at pH 1.4. The topotecan composition of Example 10 was prepared in a manner similar to that of Example 1, except that instead of HCl, 2M citric acid was used. The topotecan composition of Comparative Example 11 was prepared in a manner similar to that of Example 1, except that instead of HCl, 2M tartaric acid was used. The topotecan composition of Example 12 was prepared in a manner similar to that of Example 1, except that instead of HCl, 2M lactic acid was used. Topotecan compositions in tartaric acid showed poor physical stability compared to citric and lactic acid compositions of topotecan. Crystals were formed at the end of one month storage at 40° C. The other topotecan compositions in 2M lactic acid and citric acid at pH 1.4 showed a better stability. Also, the rate of formation of 10-HCPT is faster in tartaric acid containing topotecan formulations compared to that of hydrochloric acid and 2M lactic acid and citric acid containing topotecan formulations. Moreover, the tartaric acid containing composition also showed the formation of camptothecin which was not observed in the other formulations. The stability data are presented in Tables 11, 12 & 13 below:

### Example 9

TABLE 9
Stability data of Topotecan solution (3 mg/ml)
in 2M citric acid, pH 1.4

| | | | | Concentration | |
|---|---|---|---|---|---|
| Storage | Time | Content | % of | of 10-HCPT | |
| Temp. | Period | mg/mL | Initial | (μg/ml) | pH |
| Initial | | 3.17 | 100 | 0.32 μg/ml | 1.40 |
| 40° C. | 1 month | 3.21 | 101 | 3.49 μg/ml | 1.52 |
| | 2 months | 3.15 | 99 | 6.02 μg/ml | 1.54 |

### Comparative Example 10

TABLE 10
Stability data of Topotecan solution (3 mg/ml)
in 2M tartaric acid, pH 1.4

| | | | | Concentration | |
|---|---|---|---|---|---|
| Storage | Time | Content | % of | of 10-HCPT | |
| Temp. | Period | mg/mL | Initial | (μg/ml) | pH |
| Initial | | 3.17 | 100 | 0.32 μg/ml | 1.40 |
| 40° C. | 1 month | 3.17* | 101 | 4.76 μg/ml | 1.46 |
| | 2 months | 3.11* | 99 | 6.34 μg/ml | 1.58 |
| | | | | 0.95 μg/ml** | |

*particulate matter observed
**Camptothecin

### Example 11

TABLE 11
Stability data of Topotecan solution (3 mg/ml)
in 2M lactic acid¹, pH 1.4

| Storage | Time | Content | % of | Degradant | ID of | |
|---|---|---|---|---|---|---|
| Temp. | Period | mg/mL | Initial | Area % | Degradant | pH |
| Initial | | 3.08 | 100 | 0.06 | Unknown 1 | 1.40 |
| | | | | 0.31 μg/ml | 10-HCPT | |
| 40° C. | 1 month | 3.04 | 99 | 0.06 | Unknown 1 | 1.50 |
| | | | | 3.08 μg/ml | 10-HCPT | |
| | 2 months | 3.04 | 99 | 0.06 | Unknown 1 | 1.57 |
| | | | | 4.93 μg/ml | 10-HCPT | |
| | 3 months | 3.03 | 99 | 0.07 | Unknown 1 | 1.54 |
| | | | | 6.01 μg/ml | 10-HCPT | |

¹2M lactic acid is approximately 18%

We claim:

1. A topotecan-containing composition, comprising:
a) topotecan or a pharmaceutically acceptable salt thereof; and
b) a pharmacologically suitable fluid comprising an aqueous diluent, wherein:
i) the pH of the composition is less than or equal to about 1.5; and
ii) the composition is stable during long term storage;
wherein the amount of 10-hydroxycamptothecin (10-HCPT) in said composition resulting from the degradation of said topotecan during said long term storage is less than about 6 μg/ml.

2. A topotecan-containing composition, comprising:
a) topotecan or a pharmaceutically acceptable salt thereof; and
b) a pharmacologically suitable fluid comprising an aqueous diluent, wherein:
i) the pH of the composition is less than or equal to about 1.5; and
ii) the composition is stable during long term storage;
wherein the 10-hydroxycamptothecin (10-HCPT) resulting from the degradation of said topotecan during said long term storage does not precipitate in said pharmaceutically suitable fluid until the 10-hydroxycamptothecin (10-HCPT) reaches a concentration of about 6 μg/ml.

3. A topotecan-containing composition, comprising:
a) topotecan or a pharmaceutically acceptable salt thereof; and
b) a pharmacologically suitable fluid comprising an aqueous diluent, wherein:
i) the pH of the composition is less than or equal to about 1.2;
ii) the composition is stable during long term storage; and
iii) the topotecan concentration is about 2 mg/mL to about 4 mg/mL;
wherein the amount of 10-hydroxycamptothecin (10-HCPT) in said composition resulting from the degradation of said topotecan during said long term storage is less than about 6 μg/mL.

4. A topotecan-containing composition of claim 1 or 2, wherein the pH is less than 1.2.

5. A topotecan-containing composition of claim 1, 2 or 3, wherein the pH is between 1 and 1.2.

6. The topotecan-containing composition of claim 1, wherein the amount of formation of 10-HCPT during the shelf-life is less than about 2-4 μg/mL.

7. The topotecan-containing composition of claim 6, wherein the amount of formation 10-HCPT is less than about 1-2 μg/ml.

8. The topotecan-containing composition of claim 1, 2 or 3 wherein the long term storage is carried out a temperature of less than or equal to 25° C.

9. The topotecan-containing composition of claim 1, 2 or 3, wherein said long term storage is at least about 52 weeks.

10. The topotecan-containing composition of claim 1, 2 or 3, wherein said long term storage is at least about 78 weeks.

11. The topotecan-containing composition of claim 1, 2 or 3, wherein said long term storage is at least about 104 weeks.

12. The topotecan-containing composition of claim 1, 2 or 3, wherein the pharmacologically suitable fluid includes an acid selected from the group consisting of HCl, methane sulfonic acid, trifluoroacetic acid and mixtures thereof.

13. The topotecan-containing composition of claim 1, 2 or 3, further comprising benzyl alcohol.

14. The topotecan-containing composition of claim 1, 2 or 3, further comprising up to about 3% by weight of benzyl alcohol.

15. The topotecan-containing composition of claim 1, 2 or 3, further comprising up to about 1% by weight of benzyl alcohol.

16. A mulitdose vial containing at least 25 ml of a topotecan-containing composition of claim 1, 2 or 3, containing 2-4 mg/mL topotecan, which can be used for multiple patients for entire 28 days of chemotherapy.

17. The topotecan-containing composition of claim 1 or 2, wherein the topotecan concentration is about 1 mg/mL to about 5 mg/mL.

18. The topotecan-containing composition of claim 1 or 2, wherein the topotecan concentration is about 2 mg/mL to about 4 mg/mL.

19. The topotecan-containing composition of claim 1, 2 or 3, wherein the topotecan concentration is about 3 mg/mL.

20. A topotecan-containing composition, comprising:
a) topotecan, or a pharmaceutically acceptable salt thereof;
b) benzyl alcohol; and
c) a pharmacologically suitable fluid comprising an aqueous diluent, wherein:
i) the pH of the composition is less than or equal to about 2; and
ii) the composition is stable during long term storage, and iii) the composition is substantially free of precipitated 10-hydroxycamptothecin (10-HCPT) during said long term storage.

21. A kit comprising the topotecan-containing composition of claim 1, 2, 3 or 20.

22. A method of treating of a topotecan sensitive disease in mammals, comprising administering an effective amount of a topotecan-containing composition of claim 1, 2, 3 or 20 to a mammal in need thereof.

23. A method of preventing the formation of precipitated 10-hydroxycamptothecin in topotecan-containing aqueous formulations during long term storage at room temperature, comprising adjusting the pH of said aqueous formulation containing said topotecan to a pH of less than or equal to about 1.5 prior to initiating said long term storage.

24. A topotecan-containing composition comprising:
a) topotecan or a pharmaceutically acceptable salt thereof; and
b) a pharmacologically suitable fluid comprising hydroxyl acids selected from the group consisting of hydroxy carboxylic acids and hydroxyl tricarboxylic acids, wherein:
i) the pH of the composition is less than or equal to about 1.6;
ii) the composition is stable during long term storage; and
iii) the topotecan concentration is about 2 mg/mL to about 4 mg/mL;
wherein the amount of 10-hydroxycamptothecin (10-HCPT) in said composition resulting from the degradation of said topotecan during said long term storage is less than the solubility of the 10-HCPT in that composition.

25. The composition in claim 24, wherein the hydroxyl acid is lactic acid.

26. The composition in claim 25 where the lactic acid strength is greater than 10% and pH ranges between 1 to about 1.7.
